(19) United States
(12) Patent Application Publication
(10) Pub. No.: US 2026/0073515 A1
(43) Pub. Date: Mar. 12, 2026

(54) METHOD AND SYSTEM FOR AUTOMATED PARAMETRIC MAPPING OF BRAIN METABOLISM

(71) Applicant: University of Virginia Licensing & Ventures Group, Charlottesville, VA (US)

(72) Inventors: Bijoy Kumar Kundu, Charlottesville, VA (US); Rugved Chavan, Charlottesville, VA (US)

(21) Appl. No.: 19/328,054

(22) Filed: Sep. 12, 2025

Related U.S. Application Data
(60) Provisional application No. 63/694,099, filed on Sep. 12, 2024.

(51) Int. Cl. G06T 7/00; A61B 6/00; A61B 6/03; A61B 6/50; G06T 7/11

(52) U.S. Cl. CPC G06T 7/0012; A61B 6/037; A61B 6/507; A61B 6/5217; A61B 6/5247; G06T 7/11; G06T 2207/10088; G06T 2207/10104; G06T 2207/20084; G06T 2207/30016; G06T 2207/30104

(57) ABSTRACT

Digital images are used for time-based mapping of metabolic activity within a selected anatomy of a subject, particularly within a brain of the subject. Machine learning algorithms receive magnetic resonance image (MRI) data and four-dimensional dynamic positron emission tomography (dPET) data of the brain. A tracer may be applied prior to the anatomical scanning, and the MRI data is co-registered with the dPET data. A convolutional neural network (CNN) outputs localized data frames and a probability distribution for respective localized data frames. The probability distribution corresponds to a section of the subject's anatomy, such as internal carotid arteries, being visible in each of the respective localized data frames. The chosen section of the anatomy is segmented from the visible frames and a model-corrected input function (MCIF) for blood flow is calculated to compute a Ki map that illustrates influx of the tracer into the preferred anatomical portion.

### CROSS-REFERENCE TO RELATED APPLICATIONS

This application claims priority to and the benefit of U.S. provisional patent application No. 63/694,099, filed on Sep. 12, 2024, and titled Al Framework with 3D CNN-assisted UNETR and RNN for Blood Input Compute with Partial Volume Corrections for Automated Parametric FDG Brain PET Mapping, the disclosure of which is hereby incorporated by reference herein in its entirety.

### STATEMENT OF GOVERNMENT RIGHTS

None.

### BACKGROUND

Analyzing medical images is labor intensive if done exclusively by individuals with special training to provide manual annotations and insight into the images, often by hand in real time. A need exists for automating analysis and diagnosis of medical conditions with medical images. Though some automation has been achieved by using magnetic resonance imaging (MRI) and positron emission tomography (PET), even these images still require laborious steps of reading the images and providing a complete diagnosis for the subject. Analyzing these images could be faster and more accurate with new advances in artificial intelligence and machine learning.

MRI data acquisition and PET data acquisition are generally known techniques for retrieving medical images of a subject. PET imaging often uses a tracer and tracks the tracer in the subject's body to determine how the body is working or how a certain organ is metabolizing compounds from the bloodstream. For example, PET imaging may include using fluorodeoxyglucose as the tracer. FDG-PET imaging is widely used in clinical neuroimaging for assessing metabolic activity in the brain, particularly in conditions like epilepsy, tumors, and neurodegenerative diseases. One kind of FDG-PET generates static images of the subject's body metabolizing the tracer. Static FDG-PET provides a single snapshot of a selected anatomy of the patient metabolizing the tracer, i.e., capturing metabolic activity at a specific point in time. Static FDG-PET may be used for qualitative analysis of certain procedures and treatments applied to a subject's medical condition.

Another kind of PET scanning includes dynamic positron emission tomography (dPET) with or without the tracer. dPET offers a four dimensional (4D) visualization of metabolic activity of a tracer, with the fourth dimension being time. Dynamic FDG-PET provides richer clinical information compared to static FDG-PET [9] making it a powerful tool for neurological assessments FIG. 2 shows a comparison of static PET with dynamic PET, which allows for a quantitative selections, such as a maximum acquisition window, at different points in time.

MRI and dPET techniques provide data that is amenable to numerous kinds of mathematical and computer implemented methods of analysis. In some embodiments, artificial intelligence and machine learning techniques may be used in optional embodiments of this disclosure. Machine Learning (ML) and Artificial Intelligence (AI) systems are in widespread use in customer service, marketing, and other industries, including medicine and science. Machine learning is considered a subset of more general artificial intelligence operations, and AI endeavors may utilize numerous instances of machine learning to make decisions, predict outputs, and perform human-like intelligent operations. Machine learning protocols typically involve programming a model that instantiates an appropriate algorithm for a given computing environment and training the model on a particular data set or domain with known historical results. The results are generally known outputs of many combinations of parameter values that the algorithm accesses during training. The model uses numerous statistical and mathematical operations to learn how to make logical decisions and generate new outputs based on the historical training data. Machine learning (ML) includes, but is not limited to, a number of models such as neural networks, deep learning algorithms, support vector machines, data clustering, regression models, and Monte Carlo simulations. Other models may utilize linear regression, logistic regression, support vector machines, K-means clustering, classification models such as a binary classifier or a multi-class classifier, clustering models, anomaly detection, other supervised learning models, and even combinations of one or more machine language model types. Most of these take vectors of data as inputs.

The term “artificial intelligence,” therefore, includes any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is generally a subset of AI that enables a machine to acquire knowledge by extracting patterns from raw data.

The term “representation learning” may be used as a subset of machine learning that enables a machine to automatically discover representations needed for feature detection, prediction, or classification from raw data. Representation learning techniques include, but are not limited to, autoencoders.

The term “deep learning” may also be considered a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Some machine learning models are designed for a specific data set or domain and are highly expert at handling the nuances within that narrow domain. It is with respect to these and other considerations that the various aspects of the present disclosure as described below are presented.

This disclosure combines algorithms deciphered by artificial intelligence and machine learning with currently known systems and models that gather data from a patient on a real time basis. Accordingly this disclosure can utilize sensors and medical equipment that improve a system's ability to diagnose and treat a patient.

Brackets with numerals therein refer to references cited the below disclosure.

### SUMMARY

In one embodiment, a computer implemented method allows for using digital images to map metabolic activity within a selected anatomy of a subject. A computer having a processor connected to computer memory stores software that, when executed, performs computer instruction steps of a machine learning architecture that include collecting magnetic resonance image (MRI) data of the selected anatomy of the subject, including a preferred anatomical portion of the selected anatomy. The steps also include collecting dynamic positron emission tomography (dPET) data of the selected anatomy for the subject, in the time domain, with a tracer applied to the selected anatomy of the subject. The computer co-registers MRI data frames with dPET data frames and stores a co-registered dPET volume of frames in the computer memory. The method continues by applying the co-registered MRI data frames as inputs to a three-dimensional convolutional neural network (3D-CNN) that outputs localized data frames and a probability distribution for respective localized data frames. The probability distribution corresponds to a preferred anatomical portion being visible in each of the respective localized data frames. Visible data frames are stored in the memory by the computer by selecting, from the localized data frame, those frames having a selected probability of the preferred anatomical portion being visible therein. The method continues by segmenting the preferred anatomical portions from the visible data frames and saving segmented data frames in the computer memory. A step of calculating an image derived input function (IDIF) for blood flow into the preferred anatomical portion leads to calculating a model-corrected input function (MCIF) for blood flow using the IDIF. The computer can then compute a Ki map for the preferred anatomical portions, wherein the Ki map illustrates influx of the tracer into the preferred anatomical portion.

The computer implemented method is also embodied in a system for analyzing metabolism and a computer program product for analyzing metabolism.

### BRIEF DESCRIPTION OF THE DRAWINGS

Reference will now be made to the accompanying drawings, which are not necessarily drawn to scale. The patent or application file contains at least one drawing executed in color. Copies of this patent or patent application publication with color drawing(s) will be provided by the Office upon request and payment of the necessary fee.

FIG. 1A is a high level flow chart of an example method of utilizing machine learning processes to process digital images and map metabolic activity within a selected anatomy of a subject.

FIG. 1B is a flow chart of one embodiment of a computer implemented method that may be used according to this disclosure to map metabolic activity in a subject's body.

FIG. 2 is an overview of a computing environment capable of utilizing machine learning processes to process digital images and map metabolic activity within a selected anatomy of a subject

FIG. 3A is a computer architecture diagram showing a computing system capable of implementing aspects of the present disclosure in accordance with one or more embodiments.

FIG. 3B is a computer architecture diagram showing a networking environment that allows for data communication with a computing system capable of implementing aspects of the present disclosure in accordance with one or more embodiments.

FIG. 4 is a block diagram that illustrates a system 130 including a computer system 140 and the associated Internet 11 connection upon which an embodiment may be implemented.

FIG. 5 illustrates a system in which one or more embodiments of the disclosure can be implemented using a network, or portions of a network or computers. The present disclosure may be practiced with or without a network.

FIG. 6 illustrates an embodiment that includes, but is not limited thereto, a system, method, and computer readable medium that provides for utilizing machine learning processes to process digital images and map metabolic activity within a selected anatomy of a subject.

FIG. 7 is a schematic illustration showing a comparison of static positron emission tomography (static PET) scanning versus dynamic positron emission tomography (dPET) in the time domain.

FIG. 8 illustrates a schematic showing an overall series of steps within a deep learning pipeline used to process digital images and map metabolic activity within a selected anatomy of a subject according to this disclosure.

FIG. 9 illustrates a schematic showing a data pre-processing phase of the deep learning pipeline of FIG. 8, including motion correction and image correction according to embodiments of this disclosure.

FIG. 10 illustrates a schematic showing machine learning processes used according to this disclosure to identify localized frames of image data that have respective probabilities of showing a preferred anatomical portion from a selected anatomy in a way that is sufficiently visible for further processing.

FIG. 11 illustrates a schematic showing a machine learning processes used according to this disclosure to segment preferred anatomical portions from visible frames having the sufficient probability of FIG. 10.

FIG. 12 illustrates examples of resulting segmented images including the preferred anatomical portions of FIG. 11 as disclosed herein.

FIG. 13 illustrates a schematic showing machine learning processes used according to this disclosure to calculate a model-corrected input function (MCIF) from an image-derived input function (IDIF) according to this disclosure.

FIG. 14 is an expanded view of an inset graph of FIG. 13 showing values for the model corrected input function (MCIF) as set forth in this disclosure.

FIG. 15 illustrates a schematic showing the use of a Patlak model to map a tracer in a three-dimensional image showing how a selected anatomy, i.e., a brain, metabolizes the tracer therein.

FIG. 16A shows an example output of a mapping using the methods of this disclosure on a brain imaging technique.

FIG. 16B shows the associate brain wave results of an electroencephalogram (EEG) for the mapping of FIG. 16A.

### DETAILED DESCRIPTION

In some aspects, the disclosed technology relates to systems, methods, and computer-readable medium improving insulin therapy dosing. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures.

An aspect of an embodiment of the present disclosure provides, among other things, a system, method and computer readable medium for providing deep learning methods and multimodal deep learning architectures to distinguish tumor progression in a patient's brain from tissue necrosis caused by tumor treatments.

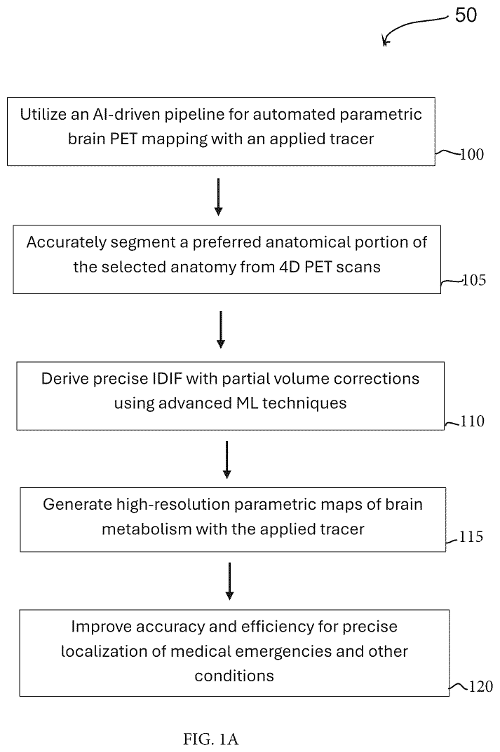

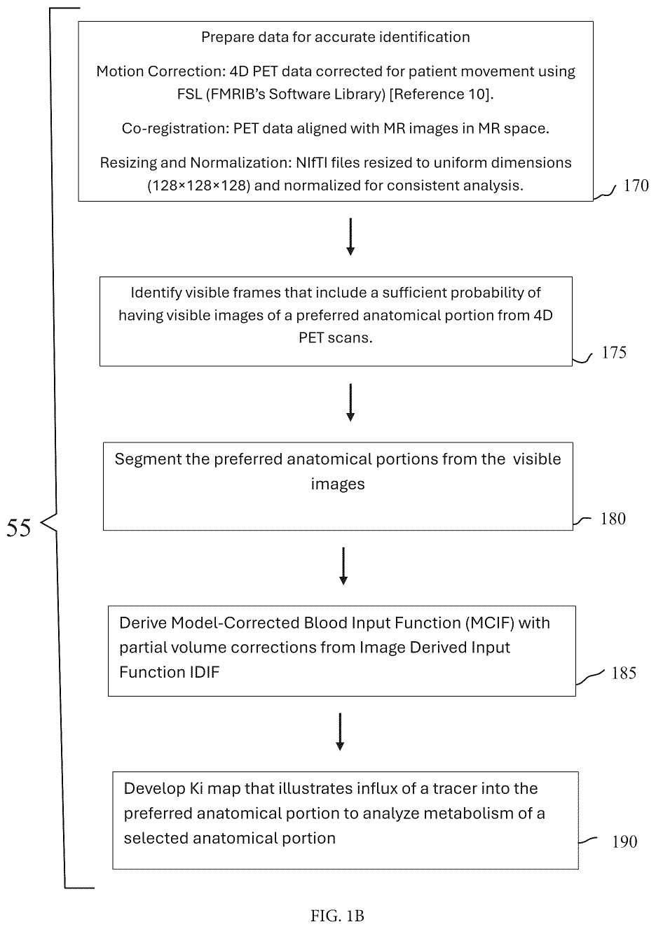

FIG. 1A illustrates a high level series of steps by which machine learning and artificial intelligence can be used to show how a selected portion of a subject's anatomy, such as a patient's brain, can metabolize compounds in the bloodstream as illustrated by a tracer progressing through the images. This disclosure utilizes steps of an AI-driven pipeline (50) for automated parametric brain PET mapping with an applied tracer (100) by accurately segmenting a preferred anatomical portion of the selected anatomy from 4D PET scans (105) and deriving precise image derived input functions (IDIF) with partial volume corrections using advanced machine language (ML) techniques (110). The method includes generating high-resolution parametric maps of brain metabolism with the applied tracer (115) to improve accuracy and efficiency for precise localization of medical emergencies and other conditions from the images (120).

FIG. 1B shows more details of a computerized method (55) according to this disclosure in which a computer and associated software can prepare data for identifying a chosen aspect of certain digital images, such as metabolism of blood components (170) and identify visible frames of data that include a sufficient probability of having visible images of a preferred anatomical portion from 4D PET scans (175). The computer implemented method continues by segmenting the preferred anatomical portions from the visible images (180). As shown in additional figures, the method is configured to derive Model-Corrected Blood Input Function (MCIF) with partial volume corrections from an Image Derived Input Function (IDIF) (185). This disclosure is therefore enabled to develop a Ki map that illustrates influx of a tracer into the preferred anatomical portion to analyze metabolism of a selected anatomical portion (190).

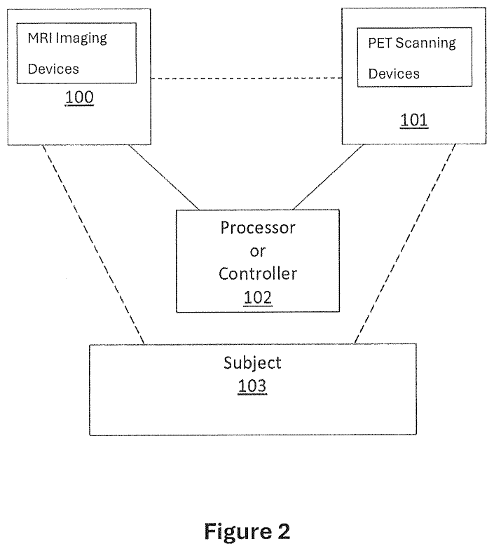

FIG. 2 is a high level functional block diagram of an embodiment of the present disclosure, or an aspect of an embodiment of the present disclosure. As shown in FIG. 2, a processor or controller 102 communicates with the glucose monitor or device 101, and optionally the insulin device 100. The glucose monitor or device 101 communicates with the subject 103 to monitor glucose levels of the subject 103. The processor or controller 102 is configured to perform the required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 can be implemented locally in the glucose monitor 101, the insulin device 100, or a standalone device (or in any combination of two or more of the glucose monitor, insulin device, or a stand along device). The processor 102 or a portion of the system can be located remotely such that the device is operated as a telemedicine device. FIG. 2 also illustrates sensors and detectors that can be used to gather field data measurements for a subject, in real time or from samples, from the patient's blood. These kinds of sensors and detectors may be stand alone equipment or incorporated into an insulin delivery device or pump.

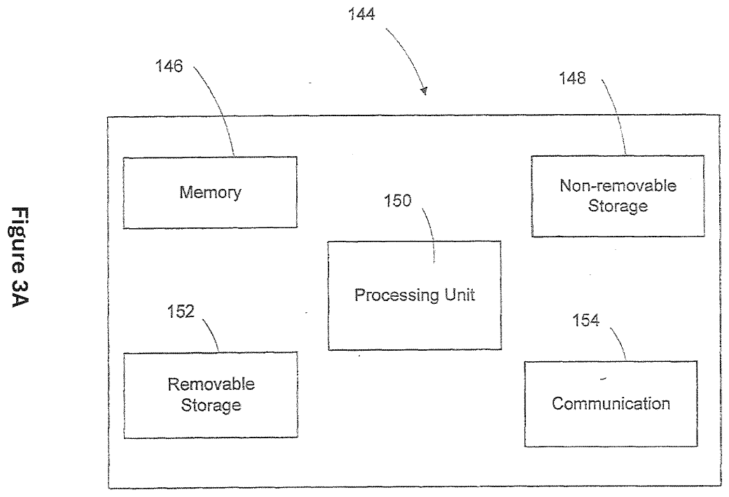

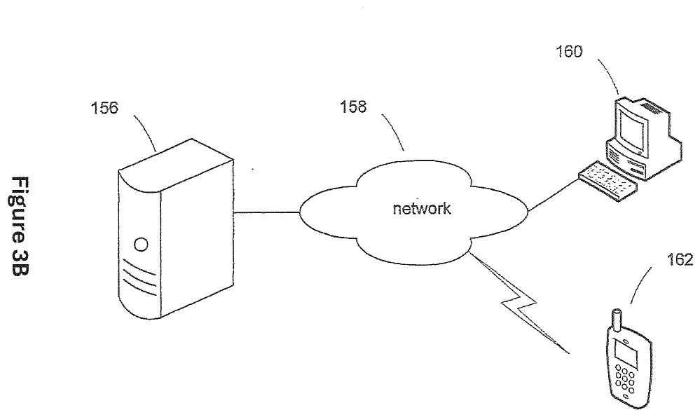

Referring to FIG. 3A, in its most basic configuration, computing device 144 typically includes at least one processing unit 150 and memory 146. Depending on the exact configuration and type of computing device, memory 146 can be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

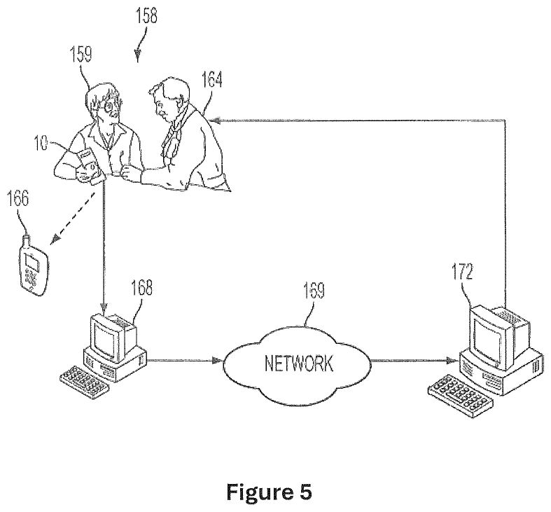

In addition to a stand-alone computing machine, embodiments of the disclosure can also be implemented on a network system comprising a plurality of computing devices that are in communication with a networking means, such as a network with an infrastructure or an ad hoc network. The network connection can be wired connections or wireless connections. As a way of example, FIG. 5 illustrates a network system in which embodiments of the disclosure can be implemented. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 3B may be multiple in number. The embodiments of the disclosure can be implemented in anyone of the devices of the system. For example, execution of the instructions or other desired processing can be performed on the same computing device that is anyone of 156, 160, and 162. Alternatively, an embodiment of the disclosure can be performed on different computing devices of the network system. For example, certain desired or required processing or execution can be performed on one of the computing devices of the network (e.g. server 156 and/or glucose monitor device), whereas other processing and execution of the instruction can be performed at another computing device (e.g. terminal 160) of the network system, or vice versa. In fact, certain processing or execution can be performed at one computing device (e.g. server 156 and/or insulin device, artificial pancreas, or glucose monitor device (or other interventional or diagnostic device)); and the other processing or execution of the instructions can be performed at different computing devices that may or may not be networked. For example, the certain processing can be performed at terminal 160, while the other processing or instructions are passed to device 162 where the instructions are executed. This scenario may be of particular value especially when the PDA 162 device, for example, accesses to the network through computer terminal 160 (or an access point in an ad hoc network). For another example, software to be protected can be executed, encoded or processed with one or more embodiments of the disclosure. The processed, encoded or executed software can then be distributed to customers. The distribution can be in a form of storage media (e.g. disk) or electronic copy.

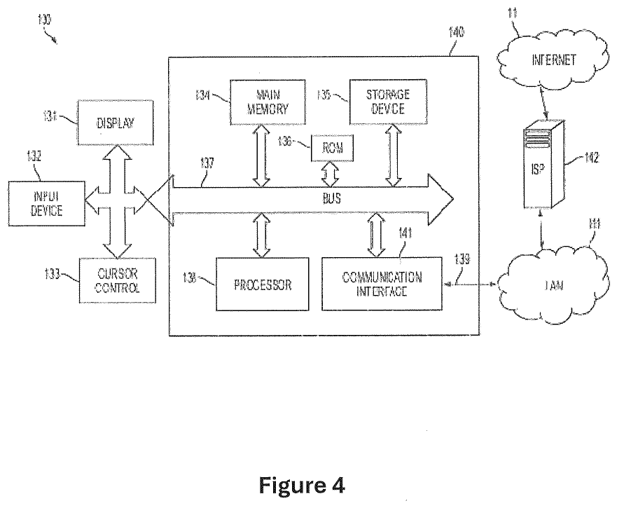

FIG. 4 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 4. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 4 illustrates various components of a computer system, it is not intended to represent any particular architecture or manner of interconnecting the components: as such details are not germane to the present disclosure. It will also be appreciated that network computers, handheld computers, cell phones and other data processing systems which have fewer components or perhaps more components may also be used. The computer system of FIG. 4 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the disclosure are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. FIG. 5 illustrates setups 158 in which multiple parties 159, 164 share information across a network 169 with numerous devices that can be a handheld telephone or mobile device 10, 166 or standard computers 168, 172. In any such implementation, communication interface 141 sends and receives electrical, electromagnetic or optical signals that carry digital data streams representing various types of information. Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world-wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

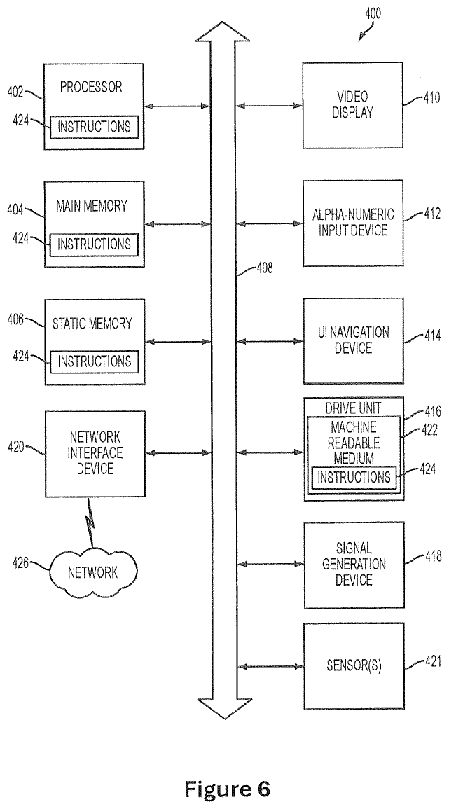

FIG. 6 is a block diagram illustrating an example of a machine upon which one or more aspects of embodiments of the present disclosure can be implemented.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices: magnetic disks such as internal hard disks and removable disks: magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

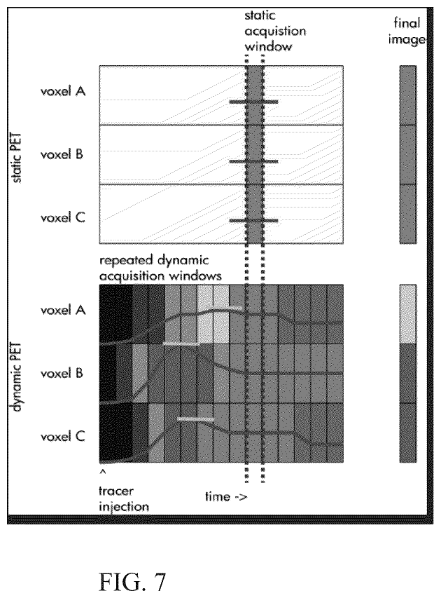

Embodiments of this disclosure take advantage of the four dimensional nature of dynamic positron emission tomography (dPET) with images being collected and synchronized in the time domain. This technology, combined with magnetic resonance imaging (MRI), provides much more precise tools to analyze activity in a selected anatomy of a patient, including but not limited to a patient's brain. FIG. 7 illustrates how much dPET can improve the availability of critical data as compared to static PET images [9].

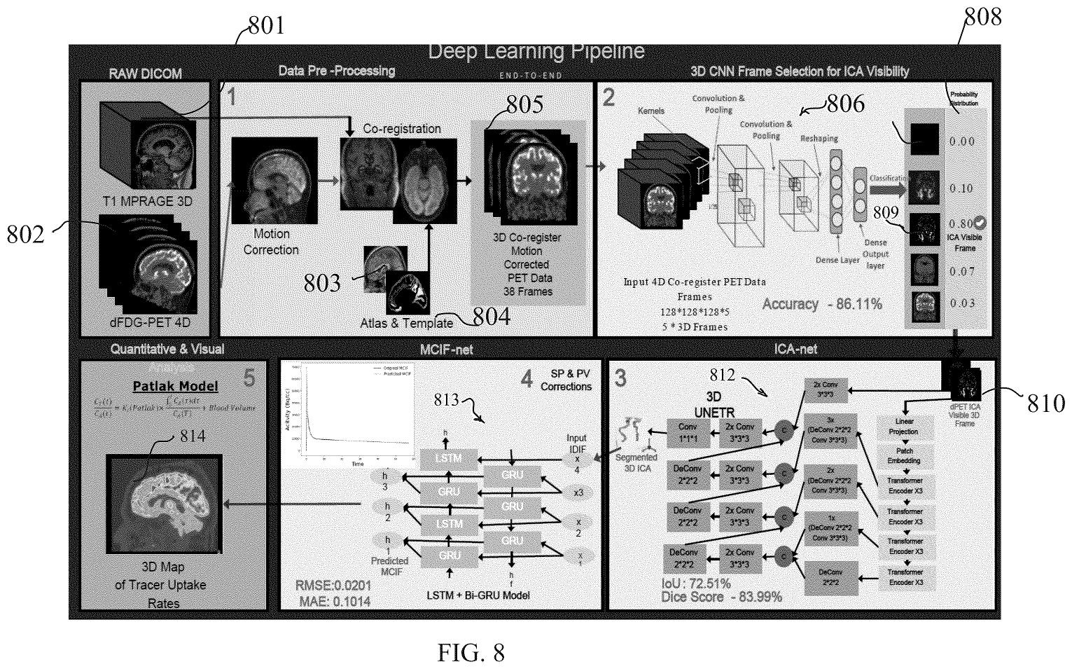

Turning to FIG. 8, one goal of this research was to develop a comprehensive end to end AI-driven pipeline for parametric fluorodeoxyglucose (FDG) brain PET mapping. The pipeline included a 3D Convolution Neural Network [1] (CNN) classifier, ‘Frame-net’ to identify internal carotid arteries (ICA) visible frames from 4D PET scans, a UNETR-based [2] ‘ICA-net’ for segmenting the ICA to derive image-derived blood input (IDIF), and a Recurrent Neural Network (RNN)-based [3] ‘MCIF-net’ to derive a model-corrected blood input function (MCIF) with partial volume (PV) corrections. FIG. 8 shows that the input images may be raw DICOM images of MRI data (e.g., TI MPRAGE 3D images (801)) and dPET data (e.g., dFDG-PET 4D images (802)). Data pre-processing (Section 1) may include motion correction and co-registration according to an appropriate atlas (803) and template (804) to form co-registered motion corrected PET data digital images (805). Section 2 of FIG. 8 shows the example artificial intelligence by which three dimensional convolutional neural networks (806) are used to output localized frames of image data showing a selected anatomy along with a probability (808) that each respective localized frame has a preferred anatomical portion (809) (e.g., internal carotid artery) that is sufficiently visible to be useful in additional steps of the analysis. Localized frames having a sufficient probability of showing visible data regarding a preferred anatomical portion can be saved as visible frames (810). Section 3 of FIG. 8 shows the artificial intelligence and machine learning algorithms that segment the visible frames (810) to show the preferred anatomical portions (809) at issue. Section 4 of FIG. 8 shows how to calculate and save in the computer memory a model corrected blood input function (MCIF) with partial volume (PV) corrections and spatial spill in (SV) corrections from an image derived input function (IDIF) with additional machine learning techniques (812, 813). Section 5 of FIG. 8 shows the resulting 3D map (814) of the tracer uptake rates for a brain image according to this disclosure.

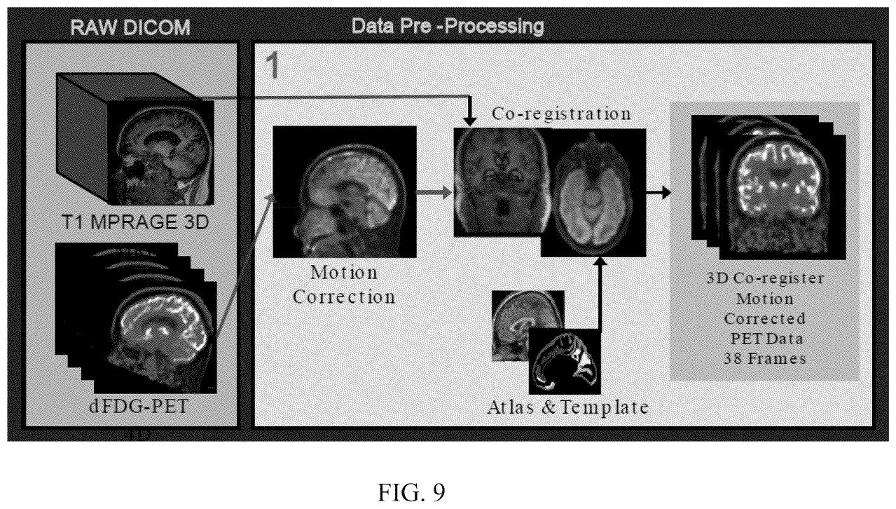

FIG. 9 illustrates an expanded view of Section 1 of FIG. 8. As shown therein, fifty (50) dynamic FDG PET brain scans using time of flight PET CT-Scanning equipment were gathered. Motion Correction included 4D PET data corrected for patient movement using FSL (FMRIB's Software Library) [10]. Co-registration was performed for the PET data to be aligned with MR images in MR space. Resizing and Normalization were applied to the NIfTI files (Neuroimaging Informatics Technology Initiative) that were resized to uniform dimensions (128×128×128) and normalized for consistent analysis.

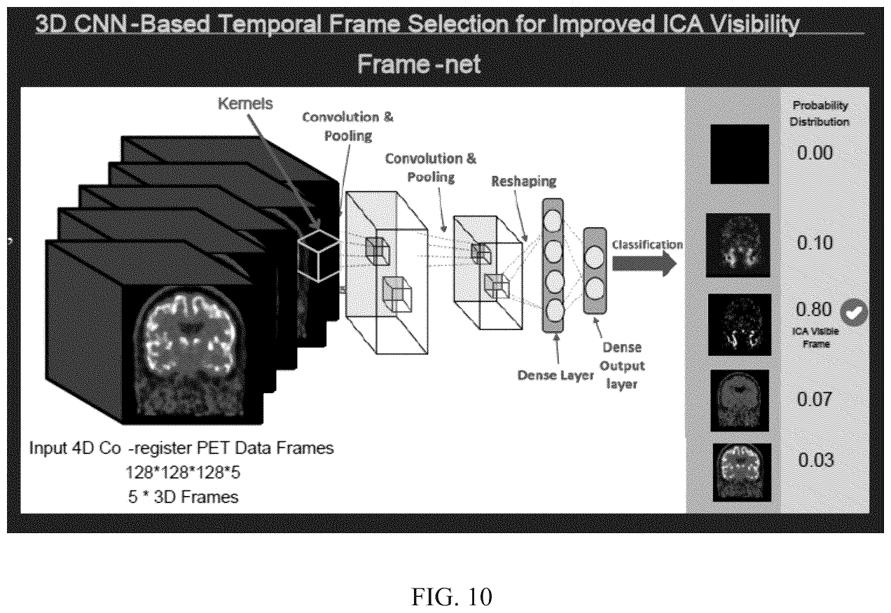

FIG. 10 illustrates an expanded view of Section 2 of FIG. 8. One objective of this example embodiment was to identify internal carotid artery (ICA) visible frames from 4D PET scans. The architecture of this example included the following, which is not limiting of this disclosure included a five channel input volume with dimensions of (128, 128, 128, 5). The convolutional layers included five layers with increasing filter sizes (64, 128, 128, 256, 256) and kernel sizes (3, 3, 3). Activation and Pooling utilized rectified linear unit (ReLU) activation and max pooling (2, 2, 2) after each convolutional layer. Dropout Layers were configured with between 0.2 and 0.3 to prevent overfitting. The Dense Layers were set to be, for example, two fully connected layers with 512 and 256 neurons, followed by “softmax” output. The training steps of FIG. 10 include 5-fold cross-validation, 100 epochs, batch size of 2 with categorical cross-entropy loss function. The evaluation metric was based on accuracy.

The outcome of FIG. 10 illustrates that Accuracy was 86.11% validation accuracy in identifying ICA-visible frames. The model was compiled using the Adam optimizer and categorical cross-entropy loss with a total 4,508,933 params and trainable params being all 4,508,933 params. Accuracy was used as the primary metric for evaluation. Training was performed over 100 epochs with a batch size of 2 to handle computational constraints. Each fold's best model, determined by the highest validation accuracy, was saved for further analysis. The 3D CNN model with temporal information rigorously tested through a 5-fold cross-validation method reached an average validation accuracy of 86.11%. 4D images, 128×128×128×5 is X and the CSV file containing numerical values for the frame number serves as Y.

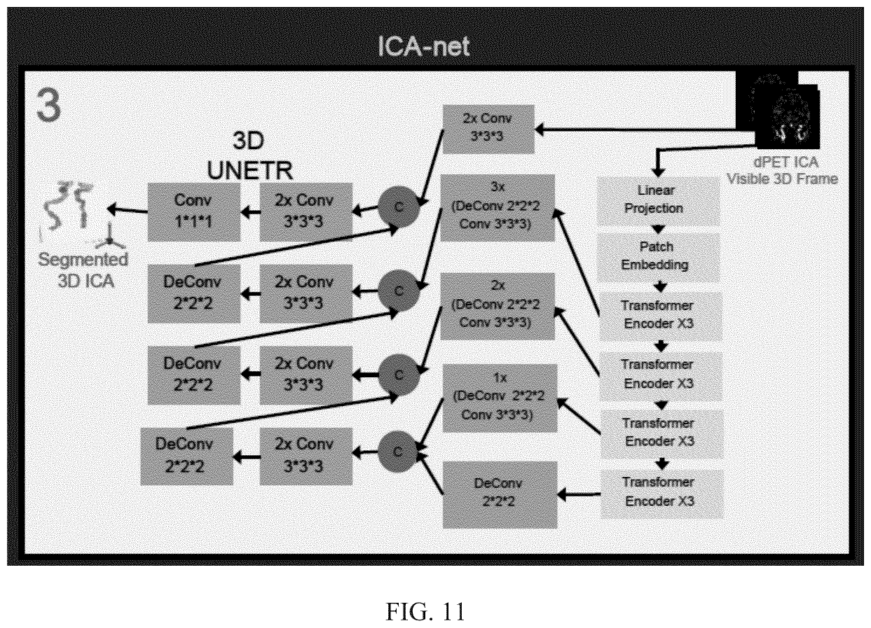

FIG. 11 illustrates more details of Section 3 of FIG. 8. One objective of this non-limiting example was to segment the internal carotid arteries (ICA) of a subject to derive image-derived blood input function (IDIF) when the subject's brain is the selected anatomy. In this section, preprocessing included resizing to the image data to 128×128×128 voxels and applying data augmentation. The non-limiting architecture shown in Section 3 of FIG. 8 only for example purposes is based on UNETR3: Optimized for 3D medical image segmentation. [2] The training, used here as one non-limiting example, was 10-fold cross-validation for robust model evaluation. The computer implemented method utilized a Loss Function of Combined Tversky and Cross-Entropy loss to address class imbalance and enhance segmentation accuracy. The Evaluation Metrics of this example for Section 3 of FIG. 8 is Dice Coefficient: 83.99% and Intersection over Union (IoU): 72.51%. The Outcome was effective segmentation of ICA, enabling accurate IDIF calculation.

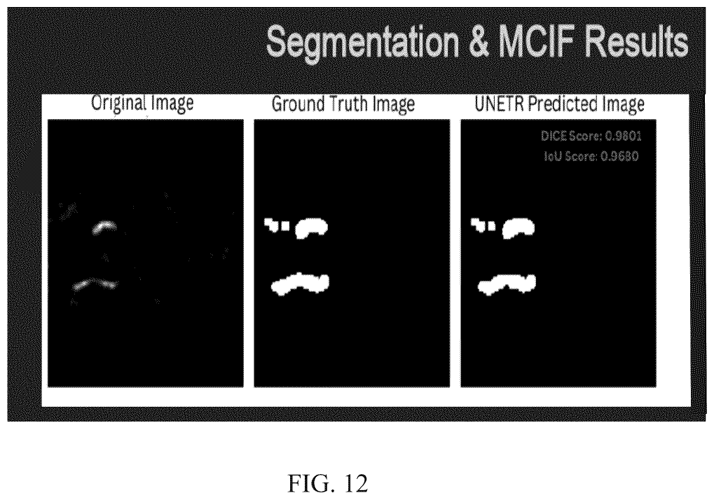

FIG. 12 is an expanded view of the results of segmenting internal carotid arteries as discussed in regard to FIG. 11. FIG. 12 shows the original image from the visible frames, and a ground truth image from a data library. The model shown in FIG. 11 resulted in a UNETR Predicted Image with a DICE Score of 0.9801 and an IoU Score of 0.9680. Validation Accuracy turned out to achieve an average validation accuracy of 86.11% in identifying ICA-visible frames from 4D PET scans. Segmentation Performance was evaluated with a DICE Coefficient of 83.99% on average across all evaluated scans. Intersection over Union (IoU) was 72.51%, demonstrating effective segmentation of the internal carotid arteries. The MCIF Section 4 of FIG. 8 relied on the segmentation of FIG. 12 with a model showing an accuracy of Root Mean Squared Error (RMSE) having a minimal error of 0.0201 in deriving the Model-Corrected Blood Input Function (MCIF).

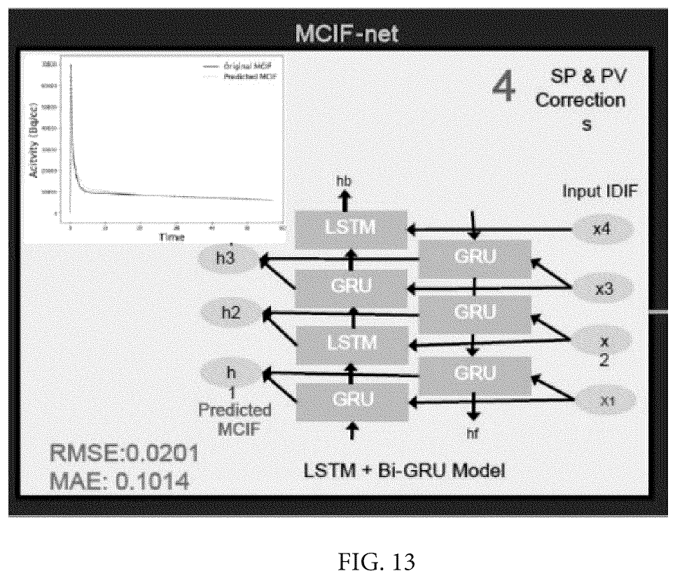

FIG. 13 illustrates more details of Section 4 of FIG. 8. One objective of this section was to derive Model-Corrected Blood Input Function (MCIF) with partial volume corrections from IDIF. An example architecture, which was not limiting of this disclosure included a Hybrid Recursive Neural Network (RNN) [11], and incorporated Long Short-Term Memory (LSTM) and Bi-directional Gated Recurrent Unit (GRU) layers to model temporal dependencies in the PET data. Dropout layers were used to prevent overfitting, and time-distributed dense layers were used to maintain temporal structure. Training incorporated 10-fold cross validation, 1000 epochs, and a batch size of 32. The Outcome for this example was a root mean square error (RMSE) with minimal error of 0.0201.

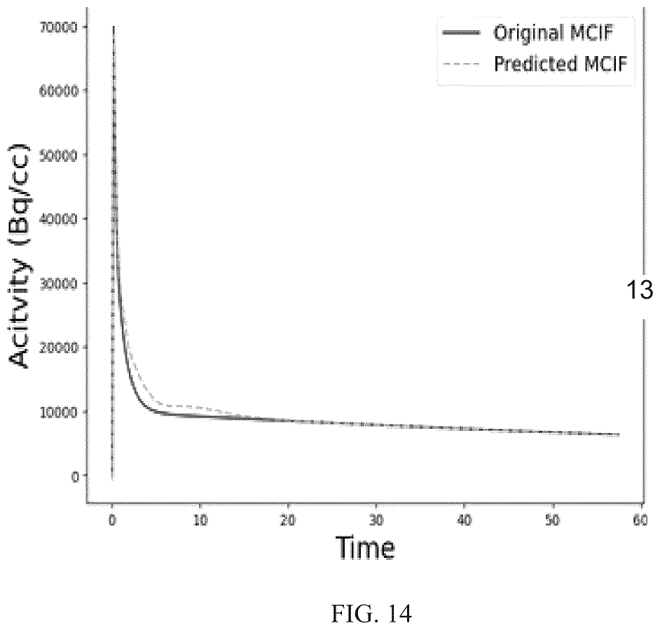

In FIG. 13, the LSTM layers, with increasing complexity from 50 to 200 neurons, were adept at capturing long-term dependencies, while the inclusion of bi-directional GRU layers provided a richer context by processing data in both forward and backward directions. Dropout layers were interspersed throughout to prevent overfitting. Time-Distributed Dense layers were incorporated to maintain the temporal structure of the output, aligning with the sequential nature of the input. The data was split according 10-fold cross validation. The system was compiled with the Adam optimizer and trained on a mean squared error loss function, and the MCIF-net underwent fine-tuning over 1000 epochs with a batch size of 32. The performance metrics (MAE and MSE) for the different architectures employed for model training indicated that a combination of Bi-directional GRU+LSTM outperformed LSTM, GRU and Bi-directional GRU alone. FIG. 14 shows an enlarged version of the graph of FIG. 13 regarding MCIF activity over time.

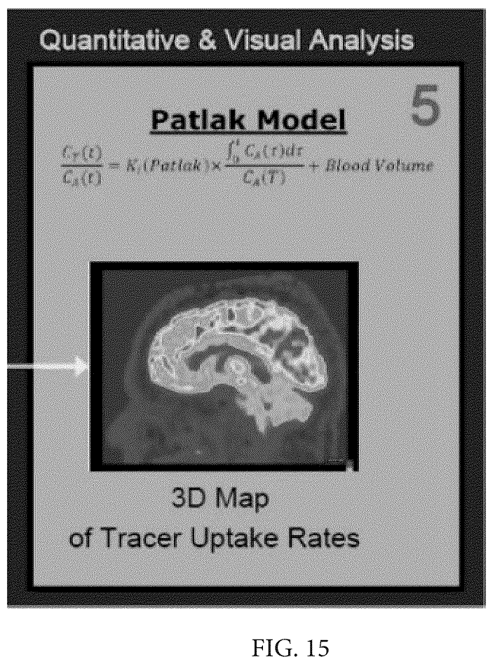

FIG. 15 shows a final application in FIG. 8, Section 4 of utilizing the MCIF to computer a Ki map with the Patlak model to show metabolism of the brain as shown.

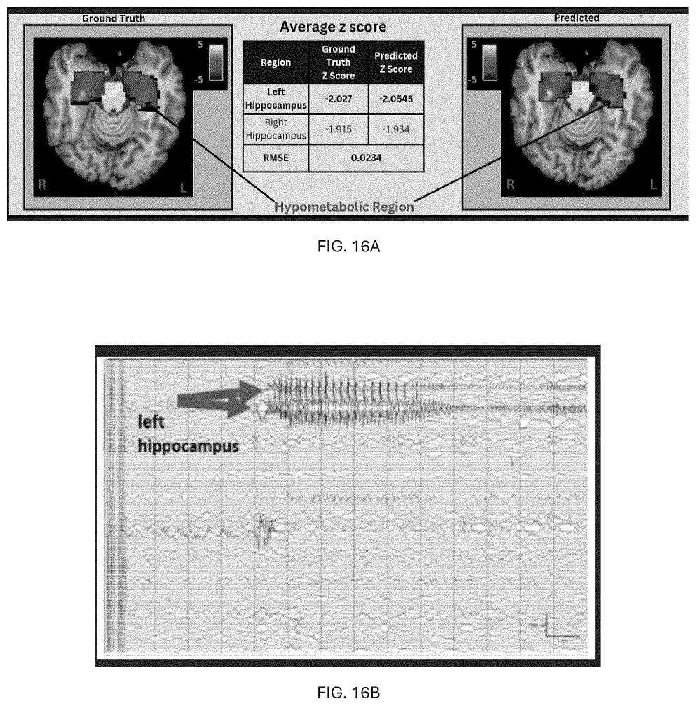

FIG. 16A is a data set showing that the computer implemented methods of this disclosure successfully identified regions of seizure onset in epilepsy subjects with known surgical ground truth. FIG. 16B illustrated an example result of treating a patient after using the methods of this disclosure. The image of FIG. 16A was performed in February 2023, and an intracranial monitoring was performed. A Laser Interstitial Thermal Therapy (LITT) procedure for epilepsy involves surgically placing a laser probe into the brain through a small hole in the skull to target and destroy the epileptic focus, which was completed in mid-July 2023 for the patient of FIG. 16A. The patient was seizure free afterward at a 2 month check in on Sep. 9, 2023.

In one embodiment, a computer implemented method allows for using digital images to map metabolic activity within a selected anatomy of a subject. A computer having a processor connected to computer memory stores software that, when executed, performs computer instruction steps of a machine learning architecture that include collecting magnetic resonance image (MRI) data of the selected anatomy of the subject, including a preferred anatomical portion of the selected anatomy. The steps also include collecting dynamic positron emission tomography (dPET) data of the selected anatomy for the subject, in the time domain, with a tracer applied to the selected anatomy of the subject. The computer co-registers MRI data frames with dPET data frames and stores a co-registered dPET volume of frames in the computer memory. The method continues by applying the co-registered MRI data frames as inputs to a three-dimensional convolutional neural network (3D-CNN) that outputs localized data frames and a probability distribution for respective localized data frames. The probability distribution corresponds to a preferred anatomical portion being visible in each of the respective localized data frames. Visible data frames are stored in the memory by the computer by selecting, from the localized data frame, those frames having a selected probability of the preferred anatomical portion being visible therein. The method continues by segmenting the preferred anatomical portions from the visible data frames and saving segmented data frames in the computer memory. A step of calculating an image derived input function (IDIF) for blood flow into the preferred anatomical portion leads to calculating a model-corrected input function (MCIF) for blood flow using the IDIF. The computer can then compute a Ki map for the preferred anatomical portions, wherein the Ki map illustrates influx of the tracer into the preferred anatomical portion.

The computer implemented method is also embodied in a system for analyzing metabolism and a computer program product for analyzing metabolism.

In one embodiment, the selected anatomy may be a brain of the subject and the preferred anatomical portion comprises an internal carotid artery (ICA) of the subject. The MRI data may be three dimensional (3D) Magnetization Prepared Rapid Gradient Echo (MP-RAGE) of 3D TI images of the selected anatomy, as shown in Section 1 of FIG. 8, and the dPET data may be four dimensional, fluorodeoxyglucose, positron emission tomography (dFDG-PET 4D). MP-RAGE data may include previously stored static MRI scans. In non-limiting embodiments, the tracer may be fluorodeoxyglucose (FDG). In the steps of co-registering the MRI data and the dPET data of FIG. 8, Section 1, the method may include aligning the MRI data and the dPET data to a template and labeling aligned data to an atlas to identify the preferred anatomical portion. The aligning may be in MRI space or dPET space. The 3D-CNN may be a neural network classifier which outputs localized data frames from which the visible data frames are selected. The visible frames are segmented with a UNETR neural network, and the method is using segmented visible data frames to calculate IDIF. Applying a Recurrent Neural Network (RNN) to the IDIF allows for deriving the MCIF with partial volume corrections. A softmax function can be used for calculating the probability distribution for respective localized data frames with a softmax function. As shown in FIG. 8, Section 2, the method allows for setting a threshold for the probability distribution, above which a respective localized data frame qualifies as a visible data frame.

### Example Embodiment and Testing

### Materials and Methods:

Dynamic FDG PET of the brain was conducted on 50 subjects using a time-of-flight PET CT scanner. Prior static MPRAGE MRI scans facilitated co-registration. Image preprocessing involved motion correction of the 4D PET data and co-registration in MR space using bash scripts designed using the FMRIB's Software Library (FSL) [4] [5].

Next, we focused on localizing the internal carotid arteries (ICA) within the early frames of the dynamic sequence using ‘Frame-net’ to identify the temporal frame displaying the ICA. For this network, five temporal frames were selected for analysis from each patient. Each NIfTI file was resized to a uniform shape of (128, 128, 128, 5) using linear interpolation and normalized. The ‘Frame-net’ architecture included an input layer with inputs of shape (128, 128, 128, 5), five convolutional layers with increasing filter sizes (64, 128, 128, 256, 256) and kernel sizes of (3, 3, 3), each followed by ReLU activation and max pooling layers (2, 2, 2), dropout Layers between 0.2 and 0.3 and two fully connected dense layers with 512 and 256 neurons, respectively, followed by a final softmax layer to output the class probabilities. The model was compiled using the Adam optimizer and categorical cross-entropy loss with a total of 4,508,933 trainable parameters. Training with 5-fold cross validation was performed over 100 epochs with a batch size of 2.

Then the selected ICA visible frames by the ‘Frame-net’ classifier were again preprocessed by resizing to 128×128×128 voxels and normalizing. Following data augmentation for both images and labels, we employed 10-fold cross validation for robust model evaluation. The ‘ICA-net’ was trained using the Adam optimizer and performance assessed using the Dice coefficient and IoU index. A combined loss function of Tversky and Cross-Entropy loss was employed to enhance sensitivity and specificity amidst class imbalance. The Tversky Loss, adjusted with alpha and beta at 0.5, and including a softmax layer for multi-class tasks, complements the Cross-Entropy Loss that refines pixel-wise classification. Losses were equally weighted (0.5 each), forming a composite loss.

Next, MCIF was computed by optimizing the IDIF obtained from the ICA [6]. The ‘MCIF-net’, mapping IDIF to MCIF, was developed using a hybrid recurrent neural network architecture incorporating both LSTM and Bi-directional GRU layers [7]. MCIF was then utilized to compute Ki Map using the Patlak model [8]. Subsequently, for an epilepsy patient with known surgical ground truth, Z-score map was computed, normalizing Ki against the mean and standard deviation (SD) for the entire brain, covering 18 super-regions per hemisphere [6].

### Results:

The Frame-net classifier reached an average validation accuracy of 86.11%. The ICA-net achieved a notable average Dice score of 83.99% and IoU of 72.51% across all evaluated scans. The MCIF-net demonstrated a minimal root mean squared error of 0.0201. When applied to actual patient data this integrated pipeline accurately identified the regions of seizure onset, leading to successful clinical interventions where the patient attained a seizure-free status post-treatment.

### Conclusion:

The efficacy of the Frame-net classifier alongside ICA-net and MCIF-net demonstrates a significant advancement in automating neuroimaging. The AI-driven pipeline marks a crucial step forward in neurological diagnostics and treatment planning.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the disclosure described herein.

It should be appreciated that any of the components or modules referred to with regards to any of the present disclosure embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

### REFERENCES

[1] F. A. Mohammed, K. K. Tune, B. G. Assefa, M. Jett, and S. Muhie, “Medical Image Classifications Using Convolutional Neural Networks: A Survey of Current Methods and Statistical Modeling of the Literature,” Mach. Learn. Knowl. Extr., vol. 6, no. 1, pp. 699-736, March 2024, doi: 10.3390/make6010033.
[2] A. Hatamizadeh et al., “UNETR: Transformers for 3D Medical Image Segmentation.” arXiv, Oct. 9, 2021. Accessed: Jun. 21, 2024. [Online]. Available: http://arxiv.org/abs/2103.10504
[3] K. Choet al., “Learning Phrase Representations using RNN Encoder-Decoder for Statistical Machine Translation.” arXiv, Sep. 2, 2014. Accessed: Jun. 21, 2024. [Online]. Available: http://arxiv.org/abs/1406.1078
[4] S. M. Smith et al., “Advances in functional and structural MR image analysis and implementation as FSL,” NeuroImage, vol. 23, pp. S208-S219, January 2004, doi: 10.1016/j.neuroimage.2004.07.051.
[5] M. W. Woolrich et al., “Bayesian analysis of neuroimaging data in FSL,” NeuroImage, vol. 45, no. 1, pp. S173-S186, March 2009, doi: 10.1016/j.neuroimage.2008.10.055.
[6] V. Seshadri et al., “Dynamic FDG-PET in localization of focal epilepsy: A pilot study,” Epilepsy Behav., vol. 122, p. 108204, September 2021, doi: 10.1016/j.yebeh.2021.108204.
[7] R. Chavan et al., “An end-to-end deep learning pipeline to derive blood input with partial volume corrections for automated parametric brain PET mapping.” arXiv, 2024. doi: 10.48550/ARXIV.2402.03414.
[8] C. S. Patlak, R. G. Blasberg, and J. D. Fenstermacher, “Graphical Evaluation of Blood-to-Brain Transfer Constants from Multiple-Time Uptake Data,” J. Cereb. Blood Flow Metab., vol. 3, no. 1, pp. 1-7, March 1983, doi: 10.1038/jcbfm.1983.1.
[9] Mark Quigg, Bijoy Kundu, Dynamic FDG-PET demonstration of functional brain abnormalities; Ann Clin Transl Neurol. 2022 Sep.;9 (9): 1487-1497. doi: 10.1002/acn3.51546. Epub 2022 Sep. 7.
[10] Jenkinson M et al, Neuroimage, 2012 Neuroimage. FSL. 2012 Aug. 15;62 (2): 782-90. doi: 10.1016/j.neuroimage.2011.09.015. Epub 2011 Sep. 16.
[11] Chavan, Rugved An end-to-end deep learning pipeline to derive blood input with partial volume corrections for automated parametric brain PET mapping; Biomed Phys Eng Express. 2024 Aug. 19;10 (5): 055028. doi: 10.1088/2057-1976/ad6a64.

1. A computer implemented method of using digital images for mapping metabolic activity within a selected anatomy of a subject, the method comprising:
using a computer having a processor connected to computer memory storing software that, when executed, performs computer instruction steps of a machine learning architecture comprising:
collecting magnetic resonance image (MRI) data of the selected anatomy of the subject including a preferred anatomical portion of the selected anatomy:
collecting dynamic positron emission tomography (dPET) data of the selected anatomy for the subject, in the time domain, with a tracer applied to the selected anatomy of the subject:
co-registering MRI data frames with dPET data frames and storing a co-registered dPET volume of frames in the computer memory:
applying the co-registered MRI data frames as inputs to a three-dimensional convolutional neural network (3D-CNN) that outputs localized data frames and a probability distribution for respective localized data frames, wherein the probability distribution corresponds to a preferred anatomical portion being visible in each of the respective localized data frames:
saving, in the computer memory, visible data frames comprising the localized data frames having a selected probability of the preferred anatomical portion being visible therein:
segmenting the preferred anatomical portions from the visible data frames and saving segmented data frames in the computer memory:
calculating an image derived input function (IDIF) for blood flow into the preferred anatomical portions:
calculating a model-corrected input function (MCIF) for blood flow using the IDIF:
computing a Ki map for the preferred anatomical portions, wherein the Ki map illustrates influx of the tracer into the preferred anatomical portion.

2. The computer implemented method of claim 1, wherein the selected anatomy comprises a brain of the subject and the preferred anatomical portion comprises an internal carotid artery of the subject.

3. The computer implemented method of claim 1, wherein the MRI data comprises three dimensional (3D) Magnetization Prepared Rapid Gradient Echo (MP-RAGE) comprising 3D Tl images of the selected anatomy, and the dPET data comprises four dimensional, fluorodeoxyglucose, positron emission tomography (dFDG-PET 4D).

4. The computer implemented method of claim 3, wherein the MP-RAGE data comprises previously stored static MRI scans.

5. The computer implemented method of claim 1, wherein the tracer comprises fluorodeoxyglucose.

6. The computer implemented method of claim 1, wherein co-registering the MRI data and the dPET data comprises aligning the MRI data and the dPET data to a template and labeling aligned data to an atlas to identify the preferred anatomical portion.

7. The computer implementation method of claim 6, wherein the aligning is in MRI space.

8. The computer implemented method of claim 1, wherein the 3D-CNN comprises a neural network classifier.

9. The computer implemented method of claim 1, further comprising segmenting the visible data frames with a UNETR neural network, and using segmented visible data frames to calculate IDIF.

10. The computer implemented method of claim 1, further comprising applying a Recurrent Neural Network (RNN) to the IDIF to derive the MCIF with partial volume corrections.

11. The computer implemented method of claim 1, further comprising calculating the probability distribution for respective localized data frames with a softmax function.

12. The computer implemented method of claim 11, further comprising setting a threshold for the probability distribution, above which a respective localized data frame qualifies as a visible data frame.

13. A system of using digital images for mapping metabolic activity within a selected anatomy of a subject, the system comprising:
a computer having a processor connected to computer memory storing software that, when executed, performs computer instruction steps of a machine learning architecture comprising:
collecting magnetic resonance image (MRI) data of the selected anatomy of the subject including a preferred anatomical portion of the selected anatomy:
collecting dynamic positron emission tomography (dPET) data of the selected anatomy for the subject, in the time domain, with a tracer applied to the selected anatomy of the subject:
co-registering MRI data frames with dPET data frames and storing a co-registered dPET volume of frames in the computer memory:
applying the co-registered MRI data frames as inputs to a three-dimensional convolutional neural network (3D-CNN) that outputs localized data frames and a probability distribution for respective localized data frames, wherein the probability distribution corresponds to a preferred anatomical portion being visible in each of the respective localized data frames:
saving, in the computer memory, visible data frames comprising the localized data frames having a selected probability of the preferred anatomical portion being visible therein:
segmenting the preferred anatomical portions from the visible data frames and saving segmented data frames in the computer memory:
calculating an image derived input function (IDIF) for blood flow into the preferred anatomical portions:
calculating a model-corrected input function (MCIF) for blood flow using the IDIF:
computing a Ki map for the preferred anatomical portions, wherein the Ki map illustrates influx of the tracer into the preferred anatomical portion.

14. The system of claim 13, further comprising a PET scanner and an MRI scanner in communication with the computer.

15. The system of claim 14, wherein the MRI scanner is configured for scanning TI images of the subject.

16. A computer program product comprising:
a non-transitory computer readable medium storing software that, when executed, performs computer instruction steps of a machine learning architecture comprising:
collecting magnetic resonance image (MRI) data of the selected anatomy of the subject including a preferred anatomical portion of the selected anatomy:
collecting dynamic positron emission tomography (dPET) data of the selected anatomy for the subject, in the time domain, with a tracer applied to the selected anatomy of the subject:
co-registering MRI data frames with dPET data frames and storing a co-registered dPET volume of frames in the computer memory:
applying the co-registered MRI data frames as inputs to a three-dimensional convolutional neural network (3D-CNN) that outputs localized data frames and a probability distribution for respective localized data frames, wherein the probability distribution corresponds to a preferred anatomical portion being visible in each of the respective localized data frames:
saving, in the computer memory, visible data frames comprising the localized data frames having a selected probability of the preferred anatomical portion being visible therein;
segmenting the preferred anatomical portions from the visible data frames and saving segmented data frames in the computer memory:
calculating an image derived input function (IDIF) for blood flow into the preferred anatomical portions:
calculating a model-corrected input function (MCIF) for blood flow using the IDIF:
computing a Ki map for the preferred anatomical portions, wherein the Ki map illustrates influx of the tracer into the preferred anatomical portion.
